(19) United States
(12) Patent Application Publication
(10) Pub. No.: US 2017/0202861 A1
(43) Pub. Date: Jul. 20, 2017

(54) Celocoxib Binding Antibodies and Uses Thereof

(71) Applicant: Autotelic LLC, City of Industry, CA (US)

(72) Inventor: Vuong Trieu, Agoura Hills, CA (US)

(73) Assignee: Autotelic LLC, City of Industry, CA (US)

(21) Appl. No.: 15/409,471

(22) Filed: Jan. 18, 2017

Related U.S. Application Data
(63) Continuation of application No. PCT/US2017/013682, filed on Jan. 16, 2017.
(60) Provisional application No. 62/279,673, filed on Jan. 16, 2016.

(51) Int. Cl. A61K 31/635; C07K 16/44; A61K 38/55; G01N 33/543; G01N 33/94

(52) U.S. Cl. CPC A61K 31/635; G01N 33/54366; G01N 33/54386; G01N 33/9486; A61K 38/556; C07K 16/44; A61K 2039/505

(57) ABSTRACT

Device and method for improving the effectiveness of osteopathic pain therapy by monitoring one or more pharmacokinetic parameters of the subject with a point-of-care device after pain drug administration. In one embodiment, the pain drug is celecoxib and the pharmacokinetic parameter is AUC.

### CROSS REFERENCE TO RELATED APPLICATIONS

The present application claims priority to U.S. Ser. No. 62/279,673, filed Jan. 16, 2016, and PCT/US2017/13682, filed Jan. 16, 2017, both herein incorporated by reference in their entirety. The following related patent applications are incorporated by reference in their entirety: PCT/US2015/034706, PCT/US2015/034708, PCT/US2015/011148, U.S. Ser. No. 14/798,753, U.S. Ser. No. 14/993,037, U.S. Ser. No. 14/798,737, U.S. Ser. No. 15/298,222 and PCT/US2016/012914.

### BACKGROUND OF THE INVENTION

The metabolism of a particular drug, and as a result, the blood concentration and the duration of action achieved by that drug, can vary significantly in a general population. (See Chun-Yu et al., Pharmacogenomics of adverse drug reactions: Implementing personalized medicine, Human Molecular Genetics, 2012, 21, Review Issue 1, B58-B65). In the care of a patient suffering from a particular disease, a drug's efficacy against diseases is a fundamental issue. However, side effects or “adverse drug reactions” (“ADRs”) caused by a drug can profoundly impact the patient, thus requiring alterations in the treatment plan. (See Lazarou et al., Incidence of adverse drug reactions in hospitalized patients: a meta-analysis of prospective studies. JAMA 1998, 279(15):1200-5). ADRs account for about 7% of all hospitalizations and consistently rank as one of the most common causes of inpatient death in western countries. (See Pirmohamed & Parle Adverse drug reactions: Back to the Future. Br. J. Clin. Pharmacol., 2003, 55, 486-492; Wester et al.: Incidence of fatal adverse drug reactions: A population based study, Br. J. Clin. Pharmacol. 2007, 65:4, 573-579).

To guard against ADRs, administering the lowest dose of a drug to a patient that achieves the greatest efficacy of the drug is of paramount importance because 75%-80% of all ADRs are dose-related (i.e., the patient experiences a side effect because they are taking too high of a dose of a particular medication). (See Routlege et al., Adverse drug reactions in elderly patients, Br J Clin Pharmacol 2004, 57:2 121-126; Lazarou et al., Incidence of adverse drug reactions in hospitalized patients: a meta-analysis of prospective studies. JAMA 1998, 279 (15):1200-5.; Melmon, K L, Morrelli, H F, Hoffman, B B, Nierenberg, D W. Melmon and Morrelli's Clinical Pharmacology: Basic Principles in Therapeutics. (3rd Edition), New York: McGraw-Hill, Inc., 1993). Despite the risks from ADRs, finding the lowest effective dose is often not addressed by conventional prescribing regimens. Additionally, the variability in individual responses to a drug significantly complicates finding the lowest effective dose. The experiences of prior patients may not be relevant to a particular individual patient's regimen. Prescribers may be deterred by a complexity of finding the lowest effective dose, and as a result, because many patients are maintained on an effective dose rather than the lowest effective dose, inadequate patient responses to the drug and/or responses with significant ADRs may occur.

Although differences in age, gender, and size contribute to the heterogeneity in drug metabolism, patients that are the same age, gender, and size can experience markedly different responses to the same drug dosage. Other factors which can influence the likelihood of ADRs include, without limitation, the administration of multiple drugs, disease state, past history of ADRs, allergic reactions, and genetic factors effecting the absorption, distribution, chemical alteration, and excretion of the drug. The ADRs from the class of drugs known as “non-steroidal anti-inflammatory drugs” (“NSAIDS”) are well documented. (See, e.g., Dieppe et al., Balancing benefits and harms: The example of non-steroidal anti-inflammatory drugs, BMJ 2004, 329, 31-34; McGettigan & Henry: Cardiovascular Risk with Non-Steroidal Anti-Inflammatory Drugs: Systematic Review of Population-Based Controlled Observational Studies, PLoS Med 2011, 8(9): e1001098. doi:10.1371/journal. pmed.1001098; Aagaard & Hansen: Information about ADRs explored by pharmacovigilance approaches: a qualitative review of studies on antibiotics, SSRIs and NSAIDs, BMC Clinical Pharmacology 2009, 9:4; Sileyman, Anti-inflammatory and side effects of cyclooxygenase inhibitors, Pharm. Reports 2007, 59, 247-58). Further, although newer NSAIDS have somewhat reduced the risk for gastrointestinal bleeding, ulceration, and perforation, they still present risks to patients such as kidney failure, hepatic dysfunction, and cardiovascular events (e.g., stroke, pain, congestive heart failure) (See CELEBREX® package insert; see also Bing, et al., Cyclooxygenase-2 inhibitors: is there an association with coronary or renal events? Current Atherosclerosis Reports 2003 5:114-7.)

The danger of NSAID ADRs is particularly important in the elderly (i.e., age >65) where drug metabolism is quite heterogeneous. (See Singh et al, Gastrointestinal Drug Interactions Affecting the Elderly, Clin. Geriatr. Med 2014, 30:1-15). As individuals age, they begin to experience diminished organ function, suffer from various diseases, and often take drugs that can interact resulting in an increased susceptibility to environmental and physical stressors (e.g., medications). As a result, seniors are generally more susceptible to the harmful side effects of NSAIDs, and yet generally receive the same dosing regimens as larger, younger individuals. (See McMillan & Hubbard: Frailty in older inpatients: What physicians need to know, Q. J. Med. 2012; 105:1059-1065; Smucker & Kontak: Adverse drug reactions causing hospital admission in an elderly population: experience with a decision algorithm, Journal of the American Board of Family Practice 1990, 3(2):105-9; Montamat et al., Management of drug therapy in the elderly, New England Journal of Medicine 1989, 321(5):303-9; and Recchia & Shear, Organization And Function Of An Adverse Drug Reaction Clinic. Journal of Clinical Psychiatry 1994, 34:68-79).

Celecoxib (sold under the brand name “CELEBREX®”) is a NSAID that has been approved for the treatment of arthritis for over 15 years. In vitro assays demonstrate that celecoxib is a potent inhibitor of prostaglandin synthesis with most of its activity resulting from its inhibition of COX-2. (See Sileyman et al., Anti-inflammatory and side effects of cyclooxygenase inhibitors, Pharm. Reports 2007, 59, 247-58). Like other NSAIDs, celecoxib puts patients, in particular elderly patients, at risk for a number of serious ADRs. For example, the 1999 CELEBREX® package insert warns that “the incidence of adverse experiences tended to be higher in elderly patients” (CELEBREX® Package Insert. Searle & Co., 1999). Similarly, the 2003 CELEBREX® package insert states that “there have been more spontaneous post-marketing reports of fatal gastrointestinal events and acute renal failure” regarding its use of in the elderly. The 2013 package insert indicates that “CELEBREX® should be used with caution in [elderly] patients.” The CELEBREX® package insert advises that for osteoarthritis and rheumatoid arthritis, the lowest effective dose of CELEBREX® should be sought for each patient.

Even the cyclooxgenase inhibitory activity of celecoxib appears to be variable (See McAdam et al., Systemic biosynthesis of prostacyclin by cyclooxygenase (COX)-2: the human pharmacology of a selective inhibitor of COX-2. PNAS. 1999; 96:272-7.) FIG. 1 is a scatterplot graph from McAdams et al. displaying the relationship between LPS-stimulated plasma PGE2 ex vivo, an index of COX-2 activity, and log plasma concentrations of celecoxib at 2, 4, 6, and 24 hours after dosing. PGE2 is expressed as a percentage of pre-dosing values. A variable dose-response is evident. (P, 0.01 vs. placebo).

It is well appreciated that non-steroidal anti-inflammatory drugs (“NSAID”) are highly active analgesics. However, NSAIDs also can have clinically significant side effects. One such side effect is drug induced edema.

“Edema” is an abnormal accumulation of fluid in the tissue spaces, cavities, or joint capsules of the body, causing swelling of the area. Edema can occur in the tissues or body spaces such as the plural cavities or the peritoneal space. Clinically, edema has variable consequences depending on the site and severity of the edema. In contrast, chronic, severe subdermal edema can cause skin break down, ulceration and serious infection. Similarly, while a pleural effusion may spontaneously resolve, ascites (edema in the peritoneal space) can be complicated by difficult to treat bacterial peritonitis. See, e.g., Harrison's Internal Medicine, 16th edition, p. 213-214.

The causes of edema are often complex. Pathophysiologically, edema occurs when one or more of the following is present elevated capillary hydraulic pressure, increased capillary permeability, and when the interstitial oncotic pressure exceeds the plasma oncotic pressure. Such changes can result from a variety of conditions and diseases. For example, in congestive heart failure the activation of the renin-angiotensin system causes volume overload which results in increased capillary hydraulic pressure. The kidneys control extracellular fluid volume by adjusting sodium and water excretion. When renal function is impaired, edema can result. In cirrhosis the reduced production of serum proteins such as albumin result in a decrease in the plasma oncotic pressure relative to interstitial oncotic pressure resulting fluid shifts into the interstitium. Venous insufficiency is a common cause of edema of the lower extremities from an increase in capillary hydraulic pressure. See, e.g., Harrison's Internal Medicine, 16^{th} edition, p. 213-214; O'Brian et al., “Treatment of Edema,” American Family Physician, 71(11). 2111-17.

Many drugs can cause edema including, without limitation, steroid hormones, vasodilators such as hydralazine, estrogens, NSAIDs, immunomodulators such as interleukin 2, and calcium channel blockers. Like other forms of edema, the pathophysiology of drug induced edema is wide ranging. Drug induced edema may be caused by vasodilation (e.g. hydralazine), drug effects on the kidneys' sodium excretion (e.g., steroids), and capillary damage (e.g., interleukin 2). Drug induced edema is usually dose-dependent and its severity increases over time. Harrison's Internal Medicine, 16th edition, p. 213-214; O'Brian et al., Treatment of Edema, American Family Physician, 71(11). 2111-17. Many NSAIDs can cause edema. The mechanism for NSAID induced edema has been postulated to be from renal vasoconstriction. Harrison's Internal Medicine, 16th edition, p. 213-214. NSAIDs inhibit cyclogenases (COX), the enzymes that catalyze formation of various prostaglandins. The two principle COX isoforms are COX-1 and COX-2. Studies have shown that both therapeutic and side effects of NSAIDs are dependent on cyclooxygenase inhibition. In general, selective inhibitors of COX-2 have therapeutic effects that are as strong as conventional NSAIDs but with fewer side effects. Nevertheless, selective COX-2 inhibitors still can cause edema. Sileyman et al., Anti-inflammatory and side effects of cyclooxygenase inhibitors, Pharma. Reports, 2007 59:247-258.

Any suitable means may be used in the detection and quantification edema varies widely. For example, effusions (edema in the thoracic, peritoneal, or pericardium) can be quantified based on the level of fluid when imaged with the patients standing. Most commonly, edema is measured subjectively based on the ability to push into or “pit” the swollen skin.

Celecoxib (under the brand name “CELEBREX®”) is a prototypic selective COX-2 inhibitor and the first page of the CELEBREX® Package Insert lists edema as an “adverse reaction.” Table 1 of this Package Insert discloses that 2.1% of patients treated with celecoxib develop edema, as compared to 1.1%, 2.1%, 1.0%, and 3.5% for placebo, naproxen, diclofenac, and ibuprofen, respectively. Moore et al.'s review of the tolerability and rate of adverse events in clinical trials of celecoxib found that the incidence of edema at any site was usually about 3%, but in two trials the incidence of edema was 23% and 38%. Arthritis Res. & Therapy, 2005, 7(6), R644-R664, R658-59 MV.

Treatment of edema consists of reversing the underlying disorder (if possible), restricting dietary sodium to minimize fluid retention, and, usually, employing a diuretic drug. O'Brian et al., Treatment of Edema, American Family Physician, 71(11). 2111-17.

In view of the persistent problem of drug induced edema and, in particular, edema induced by drugs with known efficacy for the treatment of pain, there remains a need for better approaches to preventing and treating drug induced edema. In addition, despite progress in the art, because each of the multiple mechanisms that produce drug induced edema require a specialized treatment, there remains a need for better approaches to preventing and treating drug induced edema.

### BRIEF SUMMARY OF THE INVENTION

In one aspect, the disclosure provides an antibody or antigen-binding fragment or derivative thereof that that binds to celecoxib, e.g., monoclonal antibodies CXB3, 4, or 6 produced by hybridomas of the same name. In one embodiment, the antibody or antigen-binding fragment or derivative thereof is isolated. In one embodiment, the isolated antibody or antigen-binding fragment or derivative thereof binds to celecoxib with an affinity of at least 1×10⁻⁶ K_{D}. In one embodiment, the isolated antibody or antigen-binding fragment or derivative thereof binds to celecoxib with an affinity of at least 1×10⁻⁹ K_{D}. In one embodiment, the antibody or antigen-binding fragment or derivative has a Kₒₙ at least about 1×10⁴ and a K_{off} less than about 1×10⁻³. In one embodiment, the antibody or antigen-binding fragment or derivative specifically binds to celecoxib. In one embodiment, the antibody is a monoclonal antibody. In one embodiment, the antigen-binding fragment is an Fab fragment or an F(ab)2 fragment. In one embodiment, the antigen-binding derivative is a single-chain antibody. In one embodiment, the single-chain antibody is a single chain variable fragment (scFv) or single-chain Fab fragment (scFab). In one embodiment, the antibody or antigen-binding fragment or derivative is detectably labeled.

In another aspect, the disclosure provides a lateral flow assay device, comprising:

(a) a sample receiving zone for receiving the liquid sample;
(b) a detection reagent zone in liquid communication with the sample receiving zone and downstream in flow direction from the sample receiving zone, wherein the detection reagent zone comprises a detection reagent deposited thereon, and wherein the detection reagent comprises an anti-celecoxib antibody or antigen-binding fragment or derivative thereof as described herein labeled with a detectable reporting group; and
(c) a capture zone in liquid communication with the detection reagent zone and downstream in flow direction from the detection reagent zone; wherein the capture zone comprises first and second capture reagents immobilized thereon, the first capture reagent positioned upstream in flow direction from the second capture reagent, wherein the first capture reagent comprises a celecoxib material capable of binding the detection reagent, and wherein the second capture reagent comprises an antibody capable of binding the detection reagent.

In one embodiment, the capture zone further comprises a third capture reagent immobilized thereon at a position intermediate the first and second capture agents, wherein the third capture reagent a celecoxib material capable of binding the detection reagent. In one embodiment, wherein two or three lines can be used to generate multiple readings on the same sample allowing for increase reproducibility and expanded dynamic range.

In one embodiment, the detectable reporting group is selected from colloidal gold, latex particles, colored dyes, paramagnetic and fluorescent particles. In one embodiment, the celecoxib structure is a celecoxib antigen that competes with celecoxib for binding to the detection reagent. In one embodiment, the first capture reagent is a celecoxib protein conjugate. In one embodiment, the distance between the sample receiving zone and the first capture reagent is varied to optimize celecoxib detection sensitivity. In one embodiment, the distance between the sample receiving zone and the first capture reagent is minimized to optimize celecoxib detection sensitivity. In another embodiment, the detection reagent is antibody CXB3, 4, or 6, produced by a hybridoma of the same name.

In another aspect, the disclosure provides a method of detecting the presence of celecoxib in a sample, comprising: contacting the sample to an anti-celecoxib antibody or antigen-binding fragment or derivative thereof, as described herein, under conditions sufficient to permit binding of celecoxib in the sample with the antibody or antigen-binding fragment or derivative thereof; and detecting the binding of the celecoxib to the antibody or antigen-binding fragment or derivative thereof, thereby detecting the presence of celecoxib in the sample. In another embodiment, the detection reagent is antibody CXB3, 4, or 6, produced by a hybridoma of the same name.

In one embodiment, the method further comprises quantifying the amount of celecoxib in the sample by quantifying the amount of celecoxib that is bound to the antibody or antigen-binding fragment or derivative thereof. In one embodiment, detecting the presence of celecoxib in the sample is performed in a competitive assay format. In one embodiment, detecting the presence of celecoxib in the sample is performed in a direct or indirect sandwich assay format. In one embodiment, detecting the presence of celecoxib in the sample is performed in a lateral flow assay format. In one embodiment, the lateral flow assay format comprises:

(a) applying a liquid sample comprising celecoxib to a lateral flow assay device, the device comprising:
(i) a sample receiving zone for receiving the liquid sample;
(ii) a detection reagent zone in liquid communication with the sample receiving zone and downstream in flow direction from the sample receiving zone, wherein the detection reagent zone comprises a detection reagent deposited thereon, and wherein the detection reagent comprises the anti-celecoxib antibody or antigen-binding fragment or derivative thereof labeled with a detectable reporting group; and
(iii) a capture zone in liquid communication with the detection reagent zone and downstream in flow direction from the detection reagent zone; wherein the capture zone comprises first and second capture reagents immobilized thereon, the first capture reagent positioned upstream in flow direction from the second capture reagent, wherein the first capture reagent comprises a celecoxib material capable of binding the detection reagent, and wherein the second capture reagent comprises an antibody capable of binding the detection reagent; and
(b) allowing the sample to flow from the sample receiving zone through the detection reagent zone to provide a detection reagent with celecoxib (e.g., combination of detection agent with bound celecoxib, optionally free detection reagent, and optionally free celecoxib);
(c) allowing the detection reagent with celecoxib to flow through the capture zone, whereby the first capture reagent binds free detection reagent to provide detection reagent bound to the first capture reagent, and whereby the second capture reagent binds detection reagent with or without bound celecoxib; and
(d) observing the amount of detection reagent bound to the first capture reagent relative to the second capture reagent.

In one embodiment, the capture zone further comprises a third capture reagent immobilized thereon at a position intermediate between the first and second capture reagents, wherein the third capture reagent comprises a celecoxib material capable of binding the detection reagent.

In one embodiment, the method further comprises determining the quantity of celecoxib in the sample by quantifying the amount of detection reagent bound by the first capture reagent and the second capture reagent. In one embodiment, wherein quantifying the amount of detection reagent bound to the capture reagents comprises optical density measurement. In one embodiment, the detectable reporting group is selected from colloidal gold, latex particles, colored dyes, paramagnetic and fluorescent particles. In one embodiment, the celecoxib structure is a celecoxib antigen that competes with celecoxib for binding to the detection reagent. In one embodiment, the first capture reagent is a celecoxib-protein conjugate. In one embodiment, the distance between the sample receiving zone and the first capture reagent is varied to optimize celecoxib detection sensitivity. In one embodiment, the distance between the sample receiving zone and the first capture reagent is minimized to optimize celecoxib detection sensitivity. In one embodiment, the method further comprises observing the amount of excess detection reagent bound to the second capture reagent (control line). In one embodiment, further comprises determining the quantity of celecoxib in the sample by quantitating the amount of excess detection reagent bound to the second capture reagent. In one embodiment, the sample is a liquid biological sample. In one embodiment, the liquid biological sample is blood from a subject, wherein the subject was previously administered celecoxib or a prodrug thereof. In another embodiment, the detection reagent is antibody CXB3, 4, or 6, produced by a hybridoma of the same name.

In one embodiment, the method further comprises determining the quantity of celecoxib in the sample by quantitating the amount of detection reagent bound to the third capture agent. In one embodiment, wherein quantitating the amount of detection reagent bound to the third capture reagent comprises optical density measurement. In one embodiment, wherein two or three lines can be used to generate multiple readings on the same sample allowing for increase reproducibility and expanded dynamic range.

In another aspect, the disclosure provides a method for improving the effectiveness of osteopathic pain therapy by determining one or more pharmacokinetic parameters of the subject with a point-of-care device after administration of a pain drug, the method comprising:

(a) administering a pain drug (e.g. celecoxib) at a first dose to a subject in need of osteopathic pain therapy;
(b) determining the concentration of the antihypertensive drug in the subject's blood at one or more time points after administration to provide a set of the pain drug concentration/time data points, wherein the determination of the concentration of the pain drug is made using a device or by a method described herein;
(c) transforming the set of the pain drug concentration/time data points to provide one or more pharmacokinetic parameters; and
(d) administering the pain drug at subsequent doses to achieve a target optimal value for the one or more pharmacokinetic parameters.

In one embodiment, the one or more pharmacokinetic parameters are selected from the group consisting of time to maximum concentration (Tmax), concentration maximum (Cmax), area under the curve (AUC), clearance (CL), volume of distribution (Vd), apparent volume of distribution during the terminal phase (Vz), apparent volume of distribution during steady state (Vss) and combinations thereof. In one embodiment, the one or more pharmacokinetic parameters is area-under-the-curve (AUC).

In one embodiment, the pain drug is selected from the group consisting of COX-2 inhibitor. In one embodiment, the pain drug is celecoxib.

In one embodiment, the pain drug is a COX-2 fixed-dose combination, where the other component can either be diuretic, ARB such as olmesartan, or ACE inhibitor such as lisinopril, or hydrochlorothiazide (HCTZ)

In one embodiment, the subject is in need of treatment for osteopathic pain, and the method comprises administration of celecoxib fixed-dose combination. In one embodiment, the method comprises administration of a single dosage form that comprises celecoxib and another drug. In one embodiment, the single dosage form comprises celecoxib and HCTZ or lisinopril or olmesartan

In another aspect, the disclosure provides a kit comprising the isolated antibody or antigen-binding fragment or derivative thereof as described herein. In another embodiment, the antibody is CXB3, 4, or 6, produced by a hybridoma of the same name.

### BRIEF DESCRIPTION OF THE SEVERAL VIEWS OF THE DRAWING(S)

FIG. 1 depicts the pharmacokinetic parameters produced by different doses of celecoxib.

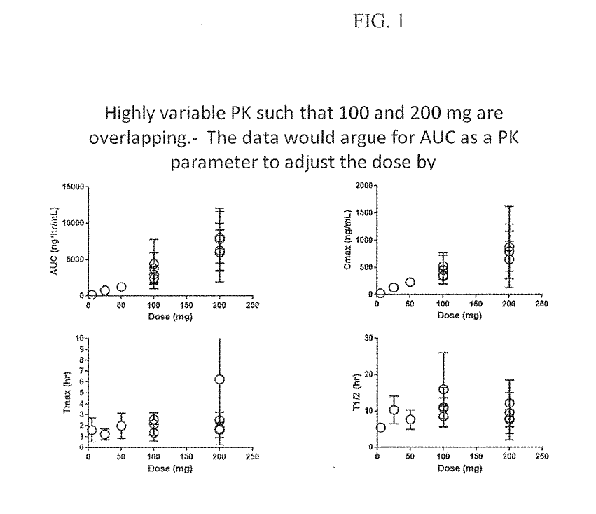

FIG. 2A is an illustration of a representative point-of-care lateral flow assay device of the invention useful for rapid therapeutic drug monitoring.

FIG. 2B is an illustration of a representative test strip for the lateral flow assay device illustrated in FIG. 2A.

FIG. 2C is an illustration of a representative test strip for the lateral flow assay device illustrated in FIG. 2A. The test strip has a single test line (T) and a single control line (C).

FIG. 2D is an illustration of a representative test strip for the lateral flow assay device illustrated in FIG. 2A. The test strip has two test lines (T1 and T2) and a single control line (C).

FIGS. 3A and 3B illustrate curves for a first representative antibody (8A10) bound at lines T1 and T2 in a representative lateral flow assay carried out with a device of the invention using the representative test strip illustrated in FIG. 2C. FIG. 3A illustrates the standard curve, i.e., the ratio of test line over control line (TIC) vs. drug concentration. The large difference in ratio for antibody (8A10) at T1 versus T2 for the lower concentrations indicates a much higher sensitivity for this antibody when placed closer to the sample port, where concentration of analyte is likely to be higher. FIG. 3B illustrates the output intensity vs. position readout of scanned test strips as provided by a reader device.

FIGS. 4A and 4B illustrate curves for a second representative antibody (3C6) bound at lines T1 and T2 in a representative lateral flow assay carried out with a device of the invention using the representative test strip illustrated in FIG. 2C. FIG. 4A illustrates the standard curve, i.e., the ratio of test line over control line (T/C) vs. drug concentration. The relatively minor difference in ratio for the antibody (3C6) at T1 versus T2 for the lower concentrations indicates a relatively low improvement in sensitivity for this antibody would be gained for placing the antibody closer to the sample port, where concentration of analyte is likely to be higher. This antibody is characterized by location independent signal from the sample port. FIG. 4B illustrates the output intensity vs. position readout of scanned test strips as provided by a reader device.

FIGS. 5A and 5B illustrate curves for combined first and second representative antibodies (8A10 and 3C6) bound at lines T1 and T2 in a representative lateral flow assay carried out with a device of the invention using the representative test strip illustrated in FIG. 2C. FIG. 5A illustrates the standard curve, i.e., the ratio of test line over control line (T/C) vs. drug concentration. The high sensitivity of T1 and T2 was obtained by combining the two antibodies (8A10 and 3C6) in the conjugate pad. This improved the dynamic range of the assay. FIG. 5B illustrates the output intensity vs. position readout of scanned test strips as provided by reader device.

FIG. 6 depicts a dose response curve obtained from testing 13 hybridomas in an antibody down ELISA assay with celecoxib-HRP competition. From the figure, it is apparent that hybridomas 3, 5, 6 and 8 are the most sensitive antibodies.

FIG. 7 depicts the celecoxib dose response curve obtained from testing hybridomas 3, 5, 6 and 8 in a celecoxib-BSA antigen down ELISA assay.

FIG. 8 depicts the celecoxib dose response curve obtained from testing hybridomas 3, 5, 6 and 8 in a celecoxib-Protein antigen down ELISA assay.

FIG. 9 depicts a celecoxib dose response curve obtained from testing 12 anti-celecoxib antibodies in an antigen-down ELISA assay using a celecoxib-BSA coated plate. The results demonstrate that antibodies 3, 5 and 6 are the most sensitive antibodies.

FIG. 10 depicts a celecoxib dose response curve obtained from testing 12 anti-celecoxib antibodies in an antigen-down ELISA assay using a celecoxib-Protein coated plate. The results demonstrate that antibodies 3, 5 and 6 are the most sensitive antibodies.

FIG. 11A, 11B, and 11C illustrate curves for a first representative anti-celecoxib monoclonal antibody (CXB6) bound in a representative lateral flow assay with one test line (FIG. 11A) or two test lines (FIG. 11B and FIG. 11C) carried out with a device of the disclosure using the representative test strips illustrated in FIGS. 2C and 2D, respectively. The illustrated standard curves show the ratio of test line over control line (T/C) vs. drug concentration.

FIGS. 12A, 12B and 12C illustrate curves for a first representative anti-celecoxib monoclonal antibody (CXB3) bound in a representative lateral flow assay with one test line (FIG. 12A) or two test lines (FIG. 12B and FIG. 12C) carried out with a device of the disclosure using the representative test strips illustrated in FIGS. 2C and 2D, respectively. The illustrated standard curves show the ratio of test line over control line (T/C) vs. drug concentration.

FIGS. 13A, 13B and 13C illustrate curves for a first representative anti-celecoxib monoclonal antibody (CXB4) bound in a representative lateral flow assay with one test line (FIG. 13A) or two test lines (FIG. 13B and FIG. 13C) carried out with a device of the disclosure using the representative test strips illustrated in FIGS. 2C and 2D, respectively. The illustrated standard curves show the ratio of test line over control line (T/C) vs. drug concentration.

### Representative Point-of-Care Assay Methods and Devices

The present invention provides a point-of-care (POC) therapeutic drug monitoring (TDM) methods, devices, and related compositions for pharmacokinetic (PK)-guided dosing of therapeutic drugs.

In one aspect, the invention provides methods and devices for immunoassay in general, and methods and devices for immunoassay of celecoxib in particular. The methods and devices of the invention provide information useful for making adjustments to the therapeutic regime for the subject.

The assay methods and devices provided herein are described in the context of compositions, methods, and devices for the detection and monitoring of celecoxib. However, it is appreciated that the format of the described compositions, methods, and devices are not so limited, and are readily applied more generally to monitoring any analyte of choice including other osteopathic pain drugs.

The present invention provides assay methods and devices for detecting or quantifying analytes (e.g., celecoxib) in a sample.

The methods and devices can be used to assay a biological sample, such as a sample obtained from a subject (patient) that has received a therapeutic agent (e.g., celecoxib) for the treatment of a condition. The sample used in the assay is ultimately a liquid sample (e.g., blood, plasma, urine).

The methods of the invention are solid phase assays and therefore are suited for adaptation to other solid phase assay configurations. To exemplify the invention, the methods and devices are described using a lateral flow assay configuration. It will be appreciated that other solid phase assays know in the art can be configured in accordance with the present methods and devices.

Lateral flow assay methods and devices can be used in accordance with the present invention. Depending on the format of the lateral flow assay method and device, the assay reagents can be disposed in certain configurations. In such an embodiment, one reagent will act as a “detection reagent” and another reagent will act as a “capture reagent.” Within this format, the detection reagent is generally deposited on the conjugate pad at a location between the sample port and a location where the capture reagent is deposited. The detection reagent generally comprises a detectable label, whereas the capture reagent is immobilized in its location on the pad. Thus, during operation, a liquid sample introduced in the sample port can flow along the pad. The sample will come into contact with the detection reagent first, and then subsequently flow over the capture reagent.

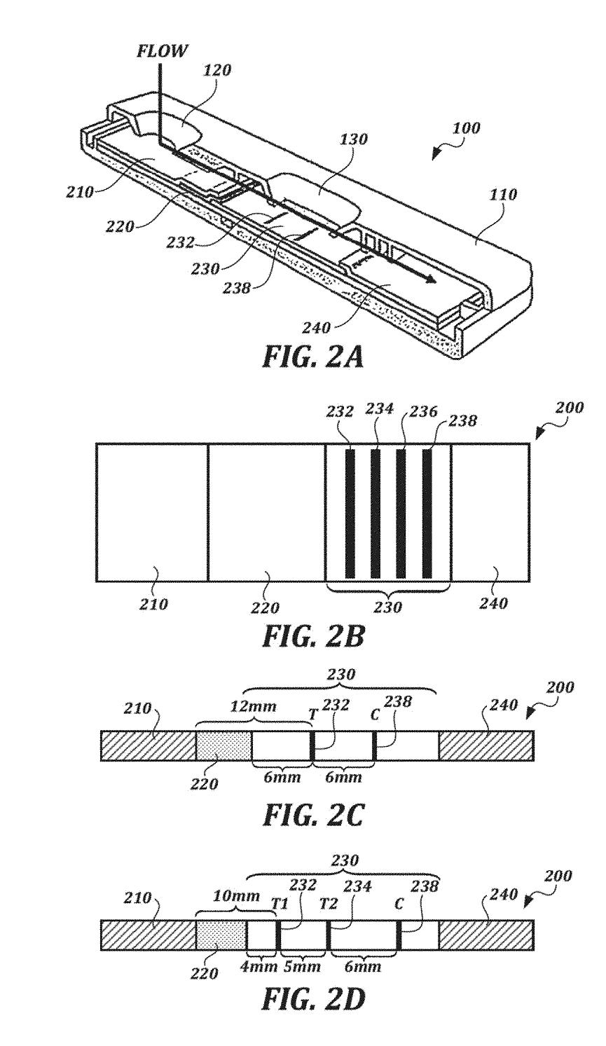

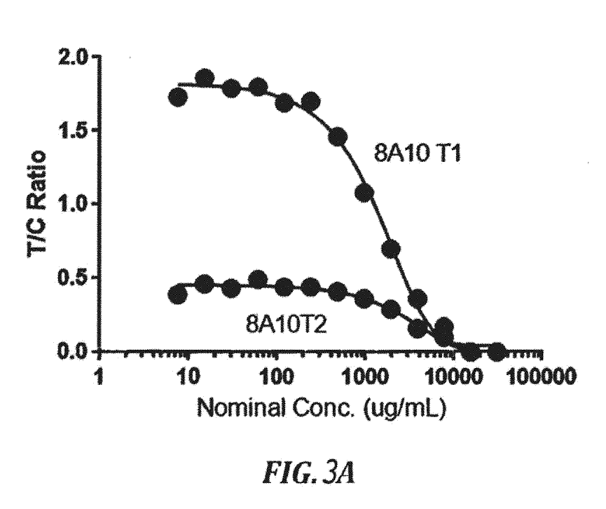

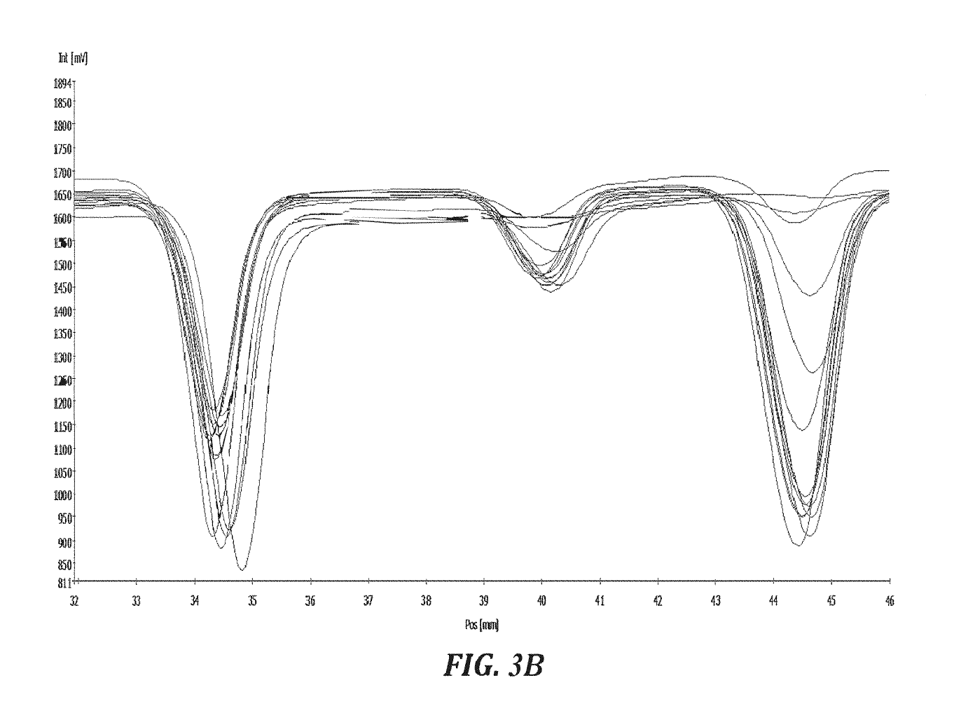

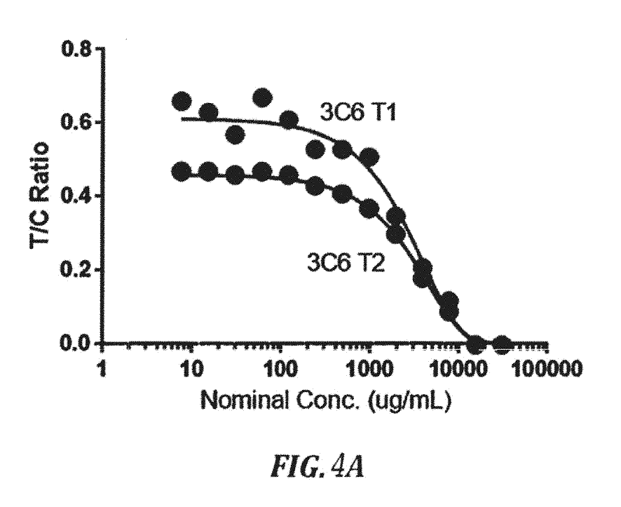

A representative device for performing a lateral flow assay in accordance with the invention is illustrated in FIG. 2A. Referring to FIG. 2A, device 100 is a cassette that includes housing 110 having sample port 120, reading window 130, and test strip 200 (see FIG. 2B). In operation, a liquid sample to be analyzed is introduced to the test strip through port 120 and is flowed along the test strip as indicated by the flow direction (from sample pad 210 to absorbent pad 240). The test results can be viewed by observing the test strip through reading window 130.

The test strip includes several zones and reagents for carrying out the assay. Referring to FIGS. 2A and 2B, representative test strip 200 includes sample pad 210, conjugate pad 220, membrane 230, and absorbent pad 240. Sample pad 210, conjugate pad 220, membrane 230, and absorbent pad 240 are in liquid communication such that liquid sample introduced to the sample pad flows through or across the conjugate pad and membrane to the absorbent pad. The size and configuration of the test strip components can be varied to suit the particular assay to be performed. For example, one or more of the component pads and membrane can overlap to facilitate optimal flow from one component to the next (sample pad 210 can overlap with conjugate pad 220, which may overlap with membrane 230, which may overlap with absorbent pad 240, as shown in FIG. 2A). The nature of the test strip zones is not particularly critical and materials for these components are known in the art.

The operation of the representative device is described as follows. Sample pad 210 receives the liquid sample to be tested. Sample flows from sample pad to conjugate pad 220.

Conjugate pad 220 includes one or more detection reagents (e.g., antibodies having an affinity for the analyte in the sample to be assayed and that are labeled to facilitate detection of the antibody in the assay).

In certain embodiments, a single detection reagent is deposited on the conjugate pad. In other embodiments, two or more detection reagents (e.g., two different antibodies, such as first and second antibodies having different affinities for the analyte to be assayed, different Kₒₙ rates, and/or different K_{off} rates) are deposited on the conjugate pad. The first and second affinities are not the same. In one embodiment, the first Kₒₙ is greater than the second Kₒₙ. In another embodiment, the second K_{off} is greater than the first K_{off}. The description and specification of antibody affinity, Kₒₙ, and K_{off} rates described below in the context of the celecoxib assay are applicable to the assay of therapeutic agents in general. The amount of first and second antibody deposited can be varied and need not be the same.

The detection reagent(s) deposited on conjugate pad 220 are mobilized by the liquid sample and flow with the sample to membrane 230. When analyte is present in the sample, binding between the analyte and detection reagent begins to occur once the sample contacts the detection reagents. Capture of the detection reagents, some of which may include bound analyte and some of which may not, occurs on membrane 230.

Membrane 230 includes at least two capture zones: a first capture zone for capturing detection reagent that does not include bound analyte (test line) (see 232 in FIGS. 2A, 2B, 2C and 2D) and a second capture zone for capturing excess detection reagent that does include bound analyte (control line) (see 238 in FIGS. 2A, 2B, 2C and 2D). The first capture zone includes a first capture material (e.g., an immobilized antigen) that is effective for capturing the detection reagent that does not include bound analyte (i.e., free detection reagent). The second capture zone includes a second capture material (e.g., an immobilized antibody) that is effective for capturing the detection reagent with or without bound analyte. The amount of detection reagent captured by the first and second capture materials, respectively, will depend on the amount of analyte present in the sample. The assay described above is a competitive assay in which the analyte and first capture material compete for affinity binding to the detection reagent. The greater the amount of analyte present in the sample, the lesser the amount of detection reagent captured by the first capture material. Due to depletion of capture material, the lesser the amount of the analyte present in the sample, the more detection reagent being capture by the first capture material and therefore less available for capture by the second capture material. The ratio of the intensity of the first and second capture lines give the best value for quantitation of the analyte.

In certain embodiments, the capture zone includes two or more first capture zones (e.g., 232 and 234 in FIGS. 2B and 2C) for capturing detection reagent that does not include bound analyte. In certain embodiments, the capture zone includes two or more second capture zones (e.g., 236 and 238 in FIG. 2B) for capturing detection reagent.

The illustrated approach of the lateral flow cassette can utilize any compatible reader with the appropriate sensitivity for detection of signal from the flow cassette and the ability to calibrate and quantify such a signal. Beneficial features of any reader can include ease of use features, including touch screen, integrated RFID or integrated barcode reader, and the capacity to easily export results, such as to a memory card or USB stick. The reader preferably has pre-installed software facilitating an interface in a selection of languages. The reader preferably has a high memory capacity to facilitate storage of multiple (such as >1000) results and can save >100 distinct test method protocols. The reader can contain connectivity to facilitate its integration into a larger system, such as through LAN or WLAN connectivity to LIS or cloud based data storage and management systems. Finally, multiple USB ports are desirable for additional connectivity capacities, such as to facilitate connection to external printers, and the like.

A representative reader is the Qiagen's Reader ESEQuant LFR (commercially available from Qiagen, Germany), which has been demonstrated as a compatible effective reader for the inclusion of the lateral flow cassette described herein. This reader is a small, portable device with internal rechargeable battery allowing it to operate out in the field and serves the requirements of the point-of-care (POC) device. The lateral flow cassette is scanned using a confocal camera system embedded in the reader. On board image analysis system is fully functional with the bar code reader of the lateral flow cassettes so that analysis method can be easily uploaded to the device.

Detection Reagents. In certain embodiments, the detection reagent is at least one antibody, antibody fragment, or antibody derivative, as described herein. The detection reagent is capable of binding the analyte in the sample (e.g., celecoxib) and when the detection reagent does not bind celecoxib in the sample, the detection reagent binds to the capture reagent

Hybridomas producing the monoclonal antibodies CXB3. Preferred detection reagents are monoclonal antibodies CXB3, CXB4, and CXB6, which bind specifically to celecoxib and are produced by hybridomas CXB3, CXB4, and CXB6 were deposited at the Budapest treaty compliant depository ______ on the date ______ and given accession numbers ______, ______, and ______. As described in the present application, the monoclonal antibodies CXB4, CXB5 and CXB6 specifically bind to celecoxib.

The detection reagents include a moiety or label that can provide a detectable signal capable of reliable quantification. Suitable moieties include those known in the immunoassay art that provide colorimetric, fluorescent, chemiluminescent, enzymatic, or radiometric signals. Representative moieties include that those provide a detectable signal that is visual and may not require instrumentation to read (e.g., colored moieties or enzymes that generate colored moieties or enzymatic. Quantitation is typically achieved through instrumental analysis of the detectable signal. In one embodiment, the detection reagent is an antibody labeled with colloidal gold, which can be visually observed.

Gold colloids are generated from reduction of gold chloride with a monodisperse nature, which are of a controlled and uniform diameter, such as 40 nm monodisperse colloid. An antibody is conjugated with colloidal gold through passive absorption.

As noted above, in preferred embodiments, multiple (i.e., more than one type of) antibodies, antibody fragments, or antibody derivatives are used. In some embodiments, the multiple (distinct) antibodies, antibody fragments, or antibody derivatives are combined and deposited in the same location on the test strip (i.e., conjugate pad).

In a representative assay for a different analyte, paclitaxel, two distinct anti-paclitaxel antibodies were used, 3C6 and 8A10. The 3C6 antibody is highly specific for paclitaxel, whereas the 8A10 antibody is less specific for paclitaxel and has a broader affinity to taxanes in general. Although, the two antibodies behave similarly in traditional competitive ELISA, it was surprisingly found that in solid phase lateral flow assays, the signal provided by 8A10 was improved by moving the first capture reagent (e.g., T1 location) closer to the sample port, as compared to 3C6, which was independent of location (T1 or T2). T1 being close to the sample application is exposed to higher concentration of the analyte, and T2 being further from the sample application is exposed to lower concentration of the analyte. This is a surprising finding that optimal placement of the capture line(s) is related to the Kₒₙ and K_{off} values of the antibodies used in the method. The availability of 3C6 allowed for construction of multiple line devices wherein the high Kₒₙ antibody (e.g., 8A10) is deposited as close to the sample origin as possible and the low K_{off} antibody (e.g., 3C6) is deposited along the pad to provide a second/third/fourth, etc., readout.

Accordingly, various modifications can be made to the lateral flow cassette device to facilitate or confer various detection properties. For example, to expand the dynamic range of the device, multiple test lines (T1, T2, etc.) with the use of multiple affinity antibodies, the dynamic range and/or the reproducibility of the assay can be expanded. The description and specification of positioning capture reagents (T/C) on the test strip described below in the context of the representative assay is applicable to positioning of capture reagents in assay of the invention in general. The preparation of representative detection reagents useful in the assays of the invention are described in Example 1.

Capture Reagents. The capture reagents serve to capture the detection reagent allowing for observation and quantitation of a detectable signal in the assay. As noted above, the assay methods and devices include first and second capture materials immobilized at first and second capture zones, respectively.

In one embodiment, the capture reagent is an immobilized analyte (e.g., celecoxib complex), which is an immobilized antigen when the detection reagent is an antibody, that captures detection reagent that does not include bound analyte. The immobilized analyte can be directly immobilized to the test strip. Alternatively, the immobilized analyte can be immobilized via a linker or carrier material (e.g., analyte conjugated to a carrier protein, such as albumin). In such an embodiment, the capture reagent is the first capture material as described above.

In one embodiment, the capture reagent is an immobilized antibody that captures detection reagent that captures detection reagent with or without bound analyte. In embodiments in which the detection reagent is a mouse monoclonal antibody, the capture reagent is an anti-mouse antibody (e.g., goat anti-mouse antibody, GAM antibody). In such an embodiment, the capture reagent is the second capture material as described above. The preparation of representative capture reagents useful in the assays of the invention are described in Example 1.

Alternative Assay Configurations. The lateral flow assay of the invention described herein is a solid phase immunoassay. It will be appreciated that the format of the assay and device can be inverted from the format described above such that the detection reagent is the labeled antigen and the capture reagent is the one or more antibody, antibody fragment, or antibody derivative (i e , immobilized in the capture zone). In the operation of such a format, the sample flows through/across the deposited labeled antigen and subsequently contacts the immobilized antibody, antibody fragment, or antibody derivative. At that point, the free analyte (e.g., celecoxib) initially present in the sample competes with the labeled antigen for binding to the immobilized antibody, antibody fragment, or antibody derivative. As above, the device can include multiple, distinct antibodies, antibody fragments, or antibody derivatives immobilized at the same or different locations. The capture reagent can be at the same or different locations. In all embodiments where the test strip has multiple locations where capture reagent is immobilized, an appropriate reader is used that can detect signal in those locations.

It is noted that the present devices, systems, compositions, and methods are generally described herein in terms of a lateral flow assay. However, the general strategy for monitoring an antigen of choice, as described herein, does not need to be limited to lateral flow assay formats, but can applied to other assay formats, such as other solid phase immunoassays (surface plasmon resonance assays), which are generally well-known in the art. Accordingly, notwithstanding description addressing lateral flow format, the present disclosure also encompasses devices, systems, compositions, and methods that incorporate any known assay format. In some embodiments, the assay format includes immobilization of capture reagents, such as the antigen conjugate (e.g., celecoxib conjugate) or antigen binding reagents (e.g., anti-celecoxib antibodies, fragments, derivatives) on a substrate. The substrate can be any known appropriate substrate for an assay format, such as nitrocellulose or glass. In some embodiments, the substrate is a nanostructure. In some embodiments, the substrate can comprise or consist of carbon nanostructures, such as carbon nanotubes, to which the capture reagents can be immobilized.

Representative Celecoxib Assay. FIG. 2C and FIG. 2D are illustrations of a representative test strip for a celecoxib lateral flow immunoassay in accordance with the invention.

Referring to FIG. 2C, representative test strip 200 includes sample pad 210, conjugate pad 220, membrane 230 with first capture zones 232 and 234 (T1 and T2) and second capture zone 238 (C), and absorbent pad 240. Referring to FIG. 2D, representative test strip 200 includes sample pad 210, conjugate pad 220, membrane 230 with first capture points 232 and 234 (T1 and T2) and second capture point 238 (C), and absorbent pad 240. As noted above with regard to FIGS. 2A and 2B, sample pad 210, conjugate pad 220, membrane 230, and absorbent pad 240 are in liquid communication such that liquid sample introduced to the sample pad flows through or across the conjugate pad and membrane to the absorbent pad; the size and configuration of the test strip components can be varied to suit the celecoxib assay to be performed (e.g., one or more of the component pads and membrane can overlap to facilitate optimal flow from one component to the next, as shown in FIG. 2A).

In one embodiment, the invention provides a method for assaying celecoxib in a liquid sample, comprising:

(a) applying a liquid sample comprising celecoxib (e.g., subject's blood sample) to a lateral flow assay device, the device having:
(i) a sample receiving zone for receiving the liquid sample;
(ii) a detection reagent zone in liquid communication with the sample receiving zone and downstream in flow direction from the sample receiving zone, wherein the detection reagent zone comprises a detection reagent deposited thereon, wherein the detection reagent is a celecoxib antibody, or fragment or derivative thereof that binds celecoxib, labeled with a detectable reporting group; and
(iii) a capture zone in liquid communication with the detection reagent zone and downstream in flow direction from the detection reagent zone, wherein the capture zone comprises first and second capture reagents immobilized thereon, the first capture reagent positioned upstream in flow direction from the second capture reagent, wherein the first capture reagent is a celecoxib material capable of binding the detection reagent, and wherein the second capture reagent is an antibody capable of binding the detection reagent;
(b) allowing the sample to flow from the sample receiving zone through the detection reagent zone to provide a detection reagent with celecoxib (e.g., combination of detection agent with bound celecoxib, optionally free detection reagent, and optionally free celecoxib);
(c) allowing the detection reagent with celecoxib to flow through the capture zone, whereby the first capture reagent binds free detection reagent to provide detection reagent bound to the first capture reagent, and whereby the second capture reagent binds detection reagent with or without bound celecoxib; and
(d) observing the amount of detection reagent bound to the first capture reagent relative to the second capture reagent.

In certain embodiments, the method further comprises determining the quantity of celecoxib in the sample by quantitating the amount of detection reagent bound to the first capture reagent. Quantitating the amount of detection reagent bound to the first capture reagent includes optical density measurements, among others.

Suitable detectable reporting groups are described above. In one embodiment, the detectable reporting group is colloidal gold.

The celecoxib antibody, or fragment or derivative thereof, useful in the present methods have a Kₒₙ greater than about 1×10⁴. Representative Kₒₙ values are greater than about 2×10⁴, 4×10⁴, 8×10⁴, 1×10⁵, 1×10⁶, and 1×10⁷). Preferred ranges are from about 1×10⁴ to about 1×10⁷.

The celecoxib antibody, or fragment or derivative thereof, useful in the present methods have a K_{off} less than about 1×10⁻³. Representative K_{off} values are less than about less than about 1×10⁻³, 1×10⁻⁴, 1×10⁻⁵, and 1×10⁻⁷. Preferred K_{off} values range from about 1×10⁻³ to 1×10⁻⁷.

In certain embodiments, the celecoxib antibody, or fragment or derivative thereof, has a Kₒₙ from about 1×10⁴ to about 1×10⁶ and a K_{off} from about 1×10⁻³ to about 1×10⁻⁴.

In one embodiment, the antibody has a high Kₒₙ and low K_{off} (e.g., minimum Kₒₙ is 2.0×10⁵ and maximum K_{off} is 1.0×10⁻³). In this embodiment, the capture line is placed at 0.0 to 0.4 T/C. For this class, monoclonal antibody engineering would focus on keeping K_{off} constant while increasing Kₒₙ as much as possible. The greater the Kₒₙ the better is the antibody detection.

In another embodiment, the antibody has a low Kₒₙ and high K_{off} (e.g., minimum Kₒₙ is 2.0×10⁴ and maximum K_{off} is 2.0×10⁻⁴. In this embodiment, the capture line is placed at 0.2-1.0 T/C. For this class, monoclonal antibody engineering would focus on keeping Kₒₙ constant while decreasing K_{off} as much as possible. The lower the off rate the better is the antibody for detection.

In the assay, the first capture zone includes an immobilized celecoxib material that serves is a celecoxib antigen that competes with celecoxib for binding to the detection reagent. The first capture zone captures detection reagent that does not include bound celecoxib (i.e., free detection reagent). In certain embodiments, the celecoxib material is a celecoxib protein conjugate. Suitable protein conjugates include serum albumin conjugates, such as BSA-celecoxib.

In the assay, the second capture zone includes an immobilized antibody capable of binding the detection reagent. In certain embodiments, the antibody is a goat anti-mouse antibody.

As noted above, the celecoxib detection sensitivity in the assay can be optimized by varying the distance between the point at which the sample is introduced to the lateral flow device (e.g., sample receiving zone) and the first capture reagent. In certain embodiments, the distance between the sample receiving zone and the first capture reagent is minimized to optimize celecoxib detection sensitivity. In certain embodiments, the distance is less than 20 mm, less than 10 mm, less than 5 mm, less than 3 mm, less than 2 mm, or less than 1 mm. In certain embodiments, the distance is from 20 to 1 mm, 10 to 1 mm, 5 to 1 mm, 3 to 1 mm, or 2 to 1 mm.

The optimization can be described as relative positioning of T (test line) and C (control line): T/C, which is defined as the distance from origin to T1/distance from origin to C ratio, where the origin is defined as the upstream edge of the capture zone (upstream edge of membrane 230 in FIGS. 2A-2D). T/C can be greater than about 0.0 (i.e., first capture reagent is located at upstream edge of capture zone), or about 0.01, about 0.02, about 0.04, about 0.08, about 0.10, about 0.20, about 0.40, about 0.80, or less than about 1.0 (i.e., first capture reagent is located at the downstream edge of the capture zone, with second capture reagent located intermediate the first capture reagent and the downstream edge of the capture zone). Preferably, T/C is from about 0.2 to about 0.7.

In certain embodiments, the ratio of the first distance to the second distance is from about 0.0 to about 0.40. In other embodiments, the ratio of the first distance to the second distance is from about 0.20 to about 1.0.

In certain embodiments, the amount of excess detection reagent that is bound to the second capture reagent is observed and measured. In certain embodiments, determining the quantity of celecoxib in the sample is determined by relating the final capture reagent (test line) to the second capture reagent (control line).

As noted above, representative assay of the invention include more than one first capture reagents in more than one first capture zone. In certain of these embodiments, the method further includes a third capture zone (see T2, 234 in FIG. 2D) intermediate the first (T1, 232 in FIG. 2D) and second (C, 238 in FIG. 2D) capture zones, wherein the third capture zone comprises a celecoxib material capable of binding the detection reagent. The celecoxib material in the first and third zones can be the same or different. In certain of these embodiments, the quantity of celecoxib in the sample is determined by quantitating the amount of detection reagent bound to the first and second capture reagents. Quantitating the amount of detection reagent bound to the first and second capture reagents can include optical density measurement.

In certain embodiments of the method, the lateral flow device further comprises an absorbent zone in liquid communication with the capture reagent zone and downstream in flow direction from the capture reagent zone.

It is noted that the methods and devices of the invention are useful for detecting levels of celecoxib in a biological sample.

The description of a representative lateral flow immunoassay in accordance with the methods and devices of the invention is described in Example 2.

### Celecoxib Antibodies

In another aspect, the invention provides antibodies (e.g., monoclonal antibodies or mAbs) that bind celecoxib. The mAbs can be purified from an antibody-rich harvested medium using MabSelect (GE Healthcare, Pittsburgh, Pa.). The mAbs can be selected based on their binding toBSA-celecoxib.

In one aspect, the invention provides an celecoxib monoclonal antibody and fragments or derivatives thereof, wherein the antibody, antibody fragment, or antibody derivative binds celecoxib. In one embodiment, the antibody is a monoclonal antibody CXB3, CXB4 or CXB6 produced by a hybridoma of the same name, accession numbers ______.

As used herein, the term “antibody” encompasses antibodies and antibody fragments thereof, derived from any antibody-producing mammal (e.g., mouse, rat, rabbit, camelid, and primate, including human) or synthetically or recombinantly produced, that specifically binds to a target of interest (e.g., celecoxib) or portions thereof. Exemplary antibodies include polyclonal, monoclonal, and recombinant antibodies; multispecific antibodies (e.g., bispecific antibodies); humanized antibodies; murine antibodies; chimeric, mouse-human, mouse-primate, primate-human monoclonal antibodies; and anti-idiotype antibodies, and may be any intact molecule or fragment thereof, such as an antigen binding fragment. As described herein, monoclonal antibodies are preferable because they provide for increased specificity in binding of the antigen of choice, such as a therapeutic drug (e.g., celecoxib).

As used herein, the term “antigen binding fragment” refers to the antigen binding or variable region from or related to a full length antibody. Illustrative examples of antibody fragments include Fab, Fab′, F(ab)2, F(ab′)2, and Fv fragments, scFv fragments, diabodies, nanobodies, linear antibodies, single chain antibody molecules, and multispecific antibodies formed from antibody fragments.

As used herein, a “single chain Fv” or “scFv” antibody fragment comprises the VH and VL domains of an antibody, wherein these domains are present in a single polypeptide chain. Generally, the Fv polypeptide further comprises a polypeptide linker between the VH and VL domains, which enables the scFv to form the desired structure for antigen binding.

As used herein, a “chimeric antibody” is a recombinant protein that contains the variable domains and complementarity-determining regions derived from a non-human species (e.g., rodent) antibody, while the remainder of the antibody molecule is derived from a human antibody.

As used herein, a “humanized antibody” is a chimeric antibody that comprises a minimal sequence that conforms to specific complementarity-determining regions derived from non-human immunoglobulin that is transplanted into a human antibody framework. Humanized antibodies are typically recombinant proteins in which only the antibody complementarity-determining regions are of non-human origin.

As used herein, the term “derivative” indicates that the antibody or antibody fragment has been produced from a reference antibody. For example, sometimes it is desirable to modify or enhance binding characteristics of a reference antibody. Thus, the antibody can be subjected to various modifications, including mutations subjected to the encoding DNA, to alter binding properties. The resulting antibody with altered properties is then referred to as a “derivative” of the reference antibody. For example, an antibody derivative can be an antibody that contains mutations resulting from affinity maturation processes that were applied to the reference antibody (or the nucleic acids encoding the reference antibody). Such mutations can result in antibodies with altered (e.g., improved) binding affinity, selectivity, and the like.

The production, processing, purification, characterization, and optimization of representative celecoxib antibodies useful in the assay methods of the invention are described in Example 3.

### Celecoxib Drug Dosing by Pharmacokinetic Parameter

In certain aspects, the invention provides methods for treating pain by administering an osteopathic pain drug in general, and celecoxib or celecoxib FDC in specific, and monitoring the subject's compliance with the prescribed dosing regimen. In the method, a dosing regimen targeting one or more specific pharmacokinetic parameters (e.g., AUC) is provided in which the one or more pharmacokinetic parameters are determined from first dosing with an osteopathic pain drug and is used to adjust subsequent dosing to achieve the targeted PK parameter. The targeted PK (AUC) dosing regimen for celecoxib was made possible by our discovery of: (1) the targeted AUC value derived from our celecoxib pharmacokinetic studies; (2) the ability to predict subsequent AUCs by AUC after first dose; and (3) the method of adjustment taking advantage of our demonstration of celecoxib dose proportionality when dosed as either celecoxib or as celecoxib FDC.

### Pain Therapy Efficacy Improvement by Monitoring Patient Compliance

In one aspect, the invention provides a method for improving the effectiveness of pain therapy by monitoring a subject's compliance by determining one or more pharmacokinetic parameters of the subject with a point-of-care device after administration of an osteopathic pain drug. In one embodiment, the method comprises:

(a) administering and osteopathic pain drug (e.g., celecoxib) at a first dose to a subject in need of pain therapy;
(b) determining the concentration of the osteopathic pain drug the subject's blood at one or more time points after administration to provide a set of osteopathic pain drug concentration/time data points, wherein the determination of the concentration of the osteopathic pain drug is made using the device of the invention described herein or by the method for assaying the osteopathic pain drug as described herein;
(c) transforming the set of osteopathic pain drug concentration/time data points to provide one or more pharmacokinetic parameters; and
(d) administering the osteopathic pain drug at subsequent doses (e.g., second and subsequent doses) to achieve a target optimal value for the one or more pharmacokinetic parameters.

Any suitable pharmacokinetic (PK) parameter or parameters can be used in accordance with this aspect of the invention, including without limiting concentration, concentration time course, peak concentration, and time after administration to peak concentration, half-life, area-under-the-curve (AUC), bioavailability, absorption, distribution, metabolism, excretion, biotransformation, or a combination thereof.

As used herein, the phrase “transforming the concentration/time data points” refers to the application of mathematical operations, formulas, theories, and/or principles (e.g., a formula for calculating AUC) to the concentrations/time data points of the individual subject to provide the pharmacokinetic value (e.g., AUC).

The target pharmacokinetic value is pre-determined by statistical analysis from a population of subjects receiving the osteopathic pain drug at its optimal dose. The term “optimal dose” refers to a dose (e.g., mg/day) associated with desirable drug efficacy at lower risk doses of a drug (e.g., the Cmax range corresponding to patients experiencing high drug efficacy at a low dose) and is determined from a statistical analysis of a subject population receiving doses of the osteopathic pain drug for whom there was therapeutic improvement without significant adverse drug reactions or significant side effects. Significant adverse drug reactions refer to ADRs that the subject finds intolerable, impair physiologic functions, and put the subject at risk for immobility and/or death or combinations thereof. Significant side effects refer to side effects that the subject finds intolerable, impair physiologic functions, and put the patient at risk for immobility and/or death or combinations thereof.

As noted above, the target pharmacokinetic parameter is the pre-determined optimal value. In certain embodiments, the target pharmacokinetic parameter is the pre-determined optimal value+/−5%. In other embodiments, the target pharmacokinetic parameter is the pre-determined optimal value+/−2%. In further embodiments, the target pharmacokinetic parameter is the pre-determined optimal value+/−1%. In yet other embodiments, the target pharmacokinetic parameter is the pre-determined optimal value+/−0.5%.

In certain embodiments, the osteopathic pain drug is celecoxib and the pharmacokinetic parameter used in the method is area-under-the-curve (AUC).

Area-under-the-curve (AUC) is a pharmacokinetic parameter that is used in the method of the invention to dose celecoxib. As used herein, the term “area under the curve (AUC)” is the area under the curve in a plot of concentration of drug in blood plasma as a function of time. Typically, the area is calculated starting at the time the drug is administered and ending when the concentration in plasma is negligible. AUC represents the total drug exposure over time. Assuming linear pharmacodynamics with elimination rate constant K, AUC is proportional to the total amount of drug absorbed by the body (i.e., the total amount of drug that reaches the blood circulation). The proportionality constant is 1/K.

For celecoxib, the target AUC of 3400 ng*hr/mL (Mean AUC of 100 mg dose) or celecoxib AUC of 6800 ng*hr/mL (Mean AUC of 200 mg dose).

Because the of dose proportionality, determination of the second dose is straightforward. When the determined pharmacokinetic (PK) parameter is the same as the target PK parameter, the second dose is the same as the first dose. When the determined PK parameter is the greater than the target, the second dose is less than the first dose by the same proportion. When the determined PK parameter is less than the target, the second dose is greater than the first dose by the same proportion.

In certain embodiments, the method further comprising repeating steps (a)-(d) until the target pharmacokinetic parameter value(s) and/or pain control is achieved.

The method of the invention is effective for monitoring compliance and treatment of an osteopathic pain drug administration regimen.

The methods of the invention are solid phase assays and therefore are suited for adaptation to other solid phase assay configurations. To exemplify the invention, the methods and devices are described using a lateral flow assay configuration. It will be appreciated that other solid phase assays know in the art can be configured in accordance with the present methods and devices.

Lateral flow assay methods and devices can be used in accordance with the present invention. Depending on the format of the lateral flow assay method and device, the assay reagents can be disposed in certain configurations. In such an embodiment, one reagent will act as a “detection reagent” and another reagent will act as a “capture reagent.” Within this format, the detection reagent is generally deposited on the conjugate pad at a location between the sample port and a location where the capture reagent is deposited. The detection reagent generally comprises a detectable label, whereas the capture reagent is immobilized in its location on the pad. Thus, during operation, a liquid sample introduced in the sample port can flow along the pad. The sample will come into contact with the detection reagent first, and then subsequently flow over the capture reagent.

A representative device for performing a lateral flow assay in accordance with the invention is illustrated in FIG. 2A. Referring to FIG. 2A, device 100 is a cassette that includes housing 110 having sample port 120, reading window 130, and test strip 200 (see FIG. 2B). In operation, a liquid sample to be analyzed is introduced to the test strip through port 120 and is flowed along the test strip as indicated by the flow direction (from sample pad 210 to absorbent pad 240). The test results can be viewed by observing the test strip through reading window 130.

The illustrated approach of the lateral flow cassette can utilize any compatible reader with the appropriate sensitivity for detection of signal from the flow cassette and the ability to calibrate and quantify such a signal. Beneficial features of any reader can include ease of use features, including touch screen, integrated RFID or integrated barcode reader, and the capacity to easily export results, such as to a memory card or USB stick. The reader preferably has pre-installed software facilitating an interface in a selection of languages. The reader preferably has a high memory capacity to facilitate storage of multiple (such as >1000) results and can save >100 distinct test method protocols. The reader can contain connectivity to facilitate its integration into a larger system, such as through LAN or WLAN connectivity to LIS or cloud based data storage and management systems. Finally, multiple USB ports are desirable for additional connectivity capacities, such as to facilitate connection to external printers, and the like.

### EXAMPLE 1

### Assay Reagents

In this example, the preparation of representative detection reagents and capture reagents useful in the assay methods and devices of the invention are described.

Detection reagents: antibody-colloidal gold conjugates. Briefly, antibodies were diluted to 1 mg/mL in 0.5×PBS and the following steps were taken: (1) shake or swirl gold to resuspend any settled gold then place 0.5 mL Naked Gold sol into 10 clean individual test tubes; (2) each tube was labeled with the pH value (or 1 through 10) from the provided pH charts; (3) pH charts were used to add varying amounts of buffer in microliters to each test tube, and shake to mix; (4) place each tube on a low speed vortexer and add the antibody solution, and mix thoroughly (about 2 to 3 seconds), for the 20 nm gold, 14 μL of a 2 mg/mL solution of antibody or protein is optimal; (5) a deepening purple color and/or black precipitate on some tubes indicate that the antibody or protein is below its isoelectric point, leading to cross-linking of individual gold solutions (cross-linked solutions cannot be used in immunological assays are discarded; deep purple solutions are mostly inactive as well; only tubes with a slight purple color or no change in color are useful for immunological assays; (6) allow the reaction to continue for a total of 30 minutes; and (7) stop the reaction by the addition of 50 μL of blocking solution.

Capture reagents: drug-albumin conjugates. Drug-albumin conjugates (e.g., BSA-paclitaxel) were prepared as described in J-G Leu et al., Cancer Res. (1993) 53:1388-1391 was generally followed.

In one exemplary embodiment, a lateral flow system was evaluated. A 0.5 mg/mL BSA-paclitaxel (Test line) and 0.5 mg/mL goat anti-mouse antibody (Control line) were striped onto the system's membrane. Paclitaxel antibody-colloidal gold conjugate was flowed through the system. The antibody-colloidal gold conjugate bound to BSA-paclitaxel immobilized on the membrane and generated a strong signal. The signal was specific to paclitaxel because a decreased signal was observed when paclitaxel was added to the spiked into the samples.

### EXAMPLE 2

### Representative Solid Phase Competitive Assay

In this example, a representative assay demonstrating the efficacy of a solid-phase competitive assay is described. The assay demonstrates the utility of using a representative antibody (anti-paclitaxel antibodies described herein) in such a detection format to provide informative signals for the present of drug in a sample. The results demonstrate that variable placement of the antibodies can enhance assay performance.

Lateral flow system. 1.2 mg/mL BSA-Pac (test lines, T) and 0.2 mg/ml of goat-anti-mouse antibody (control line, C) were striped onto a membrane card (high-flow plus HFl 80 membrane card, Millipore). Anti-paclitaxel antibody-colloidal gold conjugate was absorbed into and the dried onto a conjugate pad (glass fiber pad, Millipore). Fetal bovine serum (FBS) spiked with paclitaxel (10 μL), chased by 80 μL of PBS Tween, was flowed in the assay.

Tandem Antibody Assay. The antibody-gold conjugates are reconstituted using distilled water and are then added to each other to make the appropriate concentrations. This tandem antibody solution is applied and then dried onto the assay conjugate pads.

Reader Output: Intensity vs Position. Readout of the results of scanning the test strips. The strips were read using Qiagen reader (Qiagen, Germany).

Standard Curve. Standard curves of ratio of test line over control line vs. paclitaxel concentration were generated.

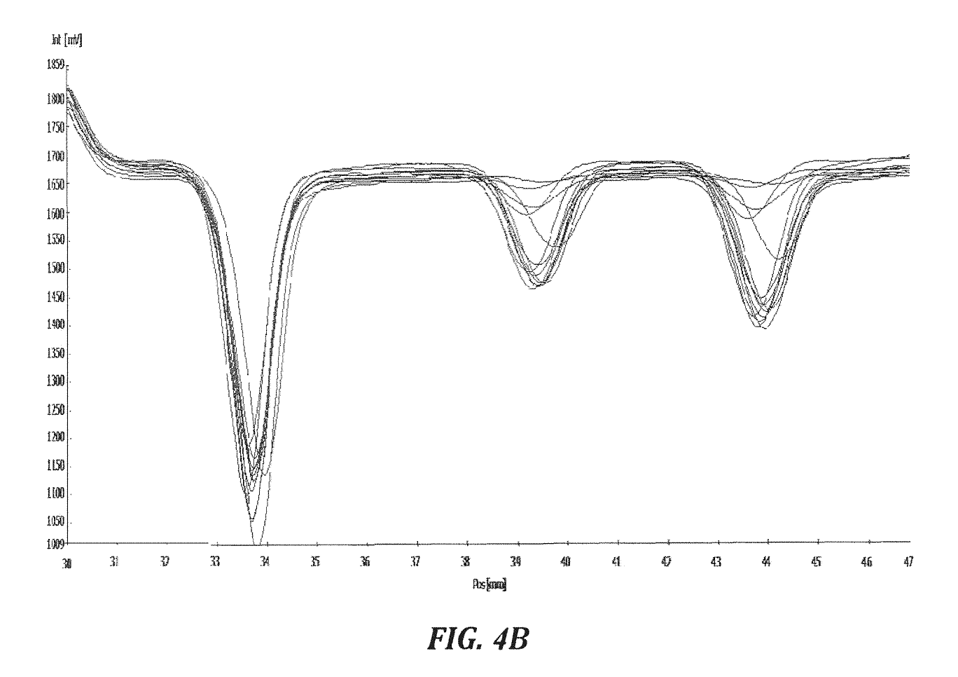

FIGS. 3A and 3B illustrate curves for 8A1 O bound at lines T1 and T2. FIG. 3A illustrates the standard curve, i.e., the ratio of test line over control line (T/C) vs. paclitaxel concentration. The large difference in ratio for 8A1 O at T1 versus T2 for the lower concentrations indicates a much higher sensitivity for the antibody when placed closer to the sample port, where concentration of analyte is likely to be higher. FIG. 3B illustrates the output intensity vs. position readout of scanned test strips as provided by a reader device.

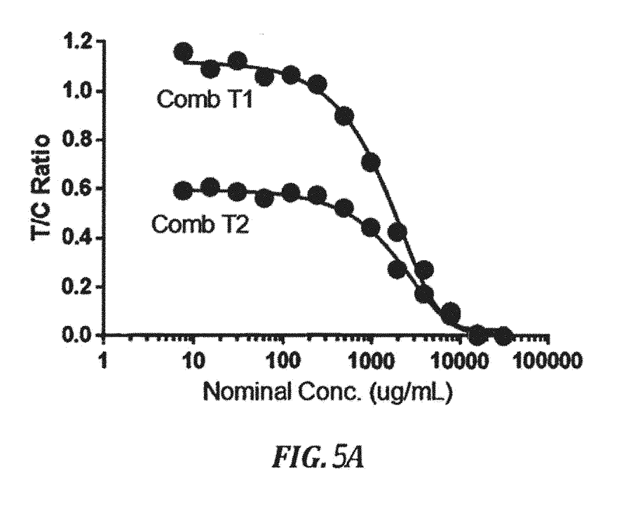

FIGS. 4A and 4B illustrate curves for 3C6 bound at lines T1 and T2. FIG. 4A illustrates the standard curve, i.e., the ratio of test line over control line (T/C) vs. paclitaxel concentration. The relatively minor difference in ratio for 3C6 at T1 versus T2 for the lower concentrations indicates a relatively low improvement in sensitivity would be gained for placing the antibody closer to the sample port, where concentration of analyte is likely to be higher. However, improvement in signal intensity relative to at T2 was observed. FIG. 4B illustrates the output intensity vs. position readout of scanned test strips as provided by a reader device.

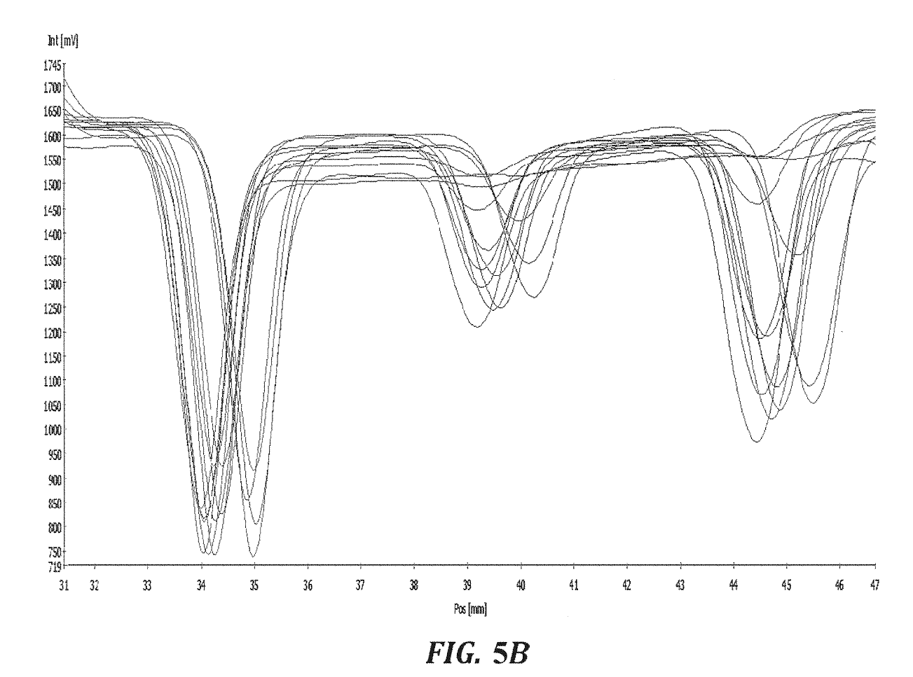

FIGS. 5A and 5B illustrate curves for combined 8A10 and 3C6 bound at lines T1 and T2. FIG. 5A illustrates the standard curve, i.e., the ratio of test line over control line (T/C) vs. paclitaxel concentration.

FIG. 5B illustrates the output intensity vs. position readout of scanned test strips as provided by reader device.

In the above analyses (and in FIGS. 3-5), the measurement of position of T1, T2, and C (Pos [mm]) in FIGS. 3B, 4B, and 5B was made from the downstream end in flow direction (e.g., sample introduced at 55 mm point, T2 at about 45 mm, T1 at about 40 mm, and Cat about 35 mm) of the test strip.

### EXAMPLE 3

### Celecoxib Antibodies

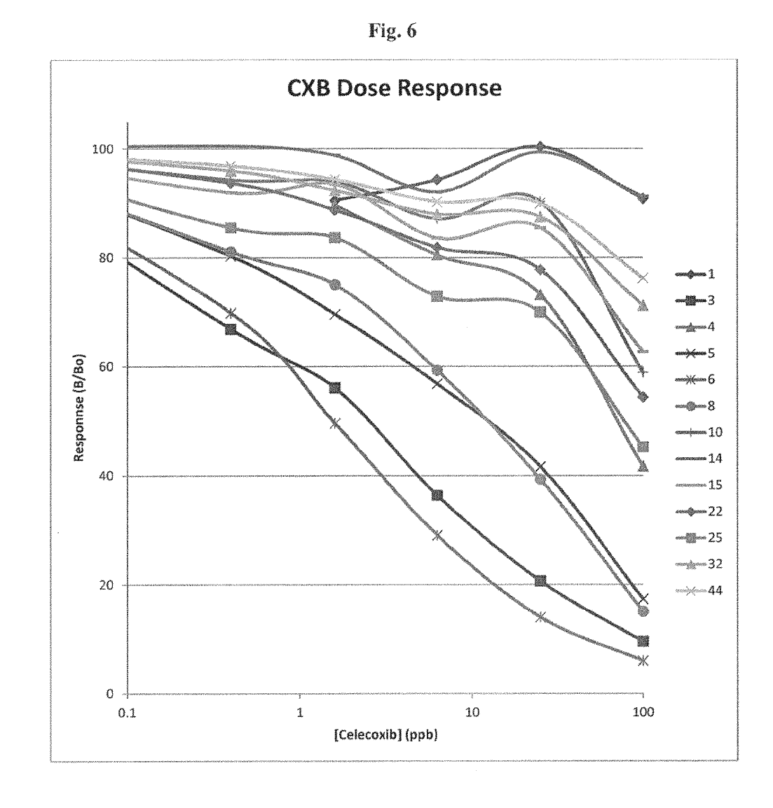

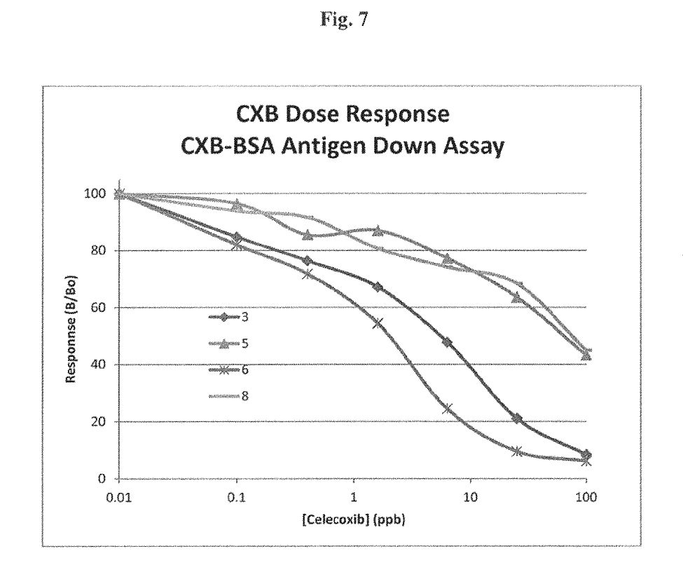

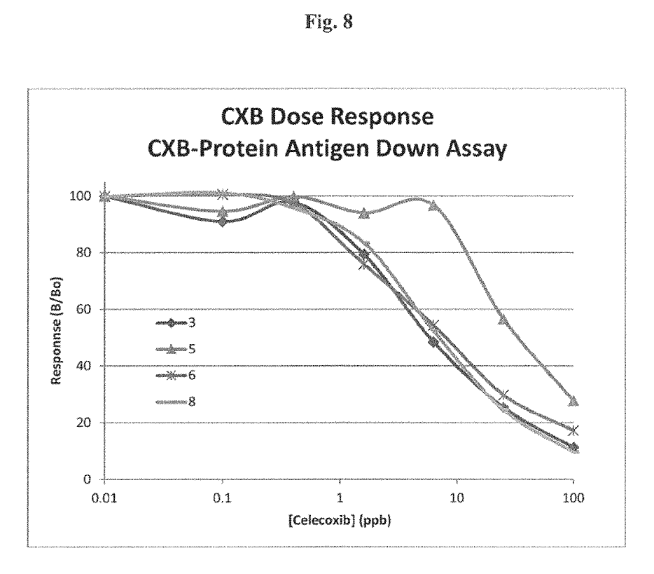

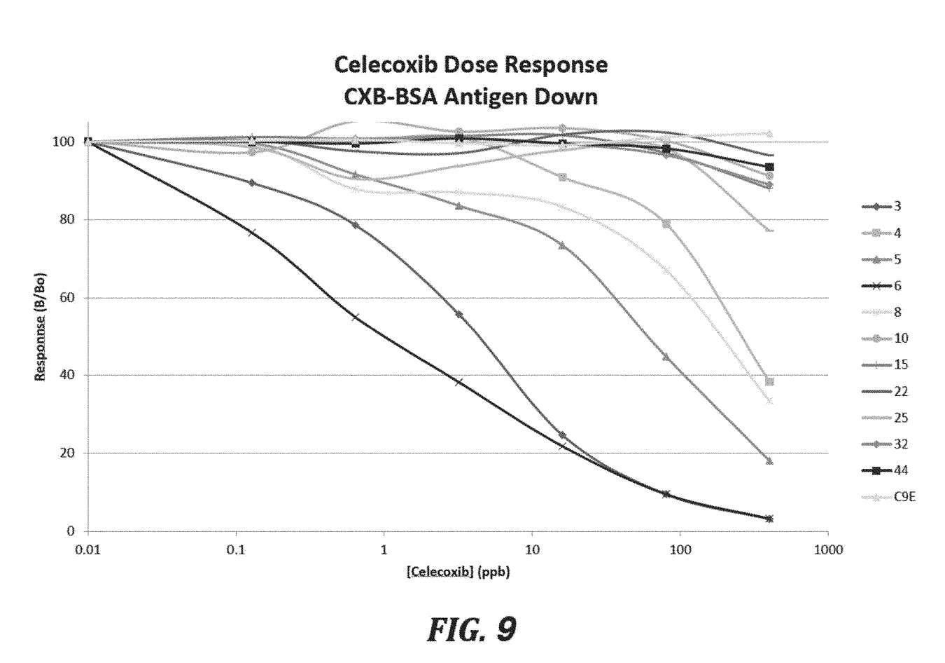

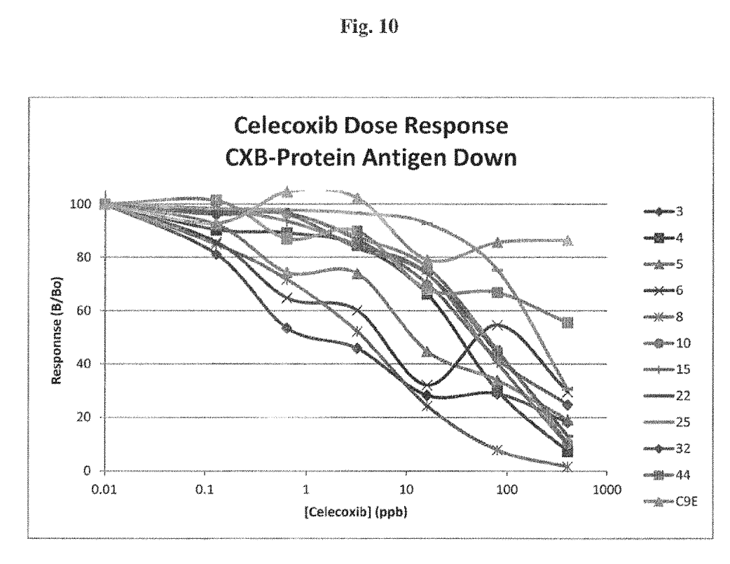

Mice were immunized with bovine serum albumin (BSA)-celecoxib conjugate. The spleens of positive mice were isolated and the antibody producing cells were used to generate a hybridoma producing monoclonal antibodies against celecoxib. The results for generated monoclonal antibodies are shown in FIGS. 6, 7, 8, 9, and 10. Thirteen hybridomas were tested in an antibody down ELISA assay with celocoxib-HRP competition. A dose response curve based on this study is presented in FIG. 6. The study demonstrated that hybridomas 3, 5, 6 and 8 were the most sensitive antibodies. These 4 antibodies were further tested in celocoxib-BSA and celocoxib-Protein antigen down Elisa assays. The binding curves obtained from these studies are presented as FIGS. 7 and 8, respectively. Twelve anti-celocoxib antibodies were tested in an antigen-down ELISA assay using a celocoxib-BSA coated plate. The results demonstrate that antibodies 3, 5 and 6 are the most sensitive antibodies. (FIG. 9) The same 12 anti-celocoxib antibodies were tested in an antigen-down ELISA assay using a celocoxib-Protein coated plate. The results confirm that antibodies 3, 5 and 6 are the most sensitive antibodies (FIG. 10).

### EXAMPLE 4

### Representative Solid Phase Competitive Assay

In this example, a representative assay demonstrating the efficacy of a solid-phase competitive assay is described. The assay demonstrates the utility of using a representative antibody (anti-celecoxib antibodies described herein) in such a detection format to provide informative signals for the presence and amount of celecoxib in a sample.

Lateral flow system. Two versions of the lateral flow format were used. The first incorporated a single test line (T) as illustrated in FIG. 2C, and the second incorporated two test lines (T1 and T2) as illustrated in FIG. 2D. The test lines of BSA-celecoxib (T, or T1 and T2) and a control line (C) of goat-anti-mouse antibody were striped onto a membrane card (high-flow plus HF180 membrane card, Millipore). The test lines T (or T1) were striped with 1.0 mg/mL BSA-celecoxib and, when tested, the additional test line T2 was striped with 0.5 mg/ml BSA-celecoxib. 0.2 mg/ml of goat-anti-mouse antibody was striped for the control (C) line.

Ruby-color colloidal gold was used to provide a signal on the agent and was made from gold(III) chloride and the pH of gold solution was adjusted to a range of pH 6.0 to pH 9.5. The specific anti-celecoxib antibody being tested was conjugated to the colloidal gold through passive absorption.

The solution of antibody-gold conjugate was directly applied to the conjugated pad while running the assay (i.e., a “liquid phase” assay). 5, 6 or 7 ul mAb-gold conjugate (OD10) was mixed with 10 ul of sample containing different amounts of celecoxib. The mixture was applied to the conjugate pad (glass fiber pad, Millipore) of the strip and chased with 80-90 ul of chasing buffer. Assay time is 15-20 mins before taking the reading.

A “solid phase” lateral flow assay can be performed by applying and drying the detection reagent (e.g., anti-celecoxib antibody conjugated with gold colloid) to the conjugate pad. For example, 8% (w/v) sucrose and 2% (w/v) trehalose are used to stabilize mAb-gold conjugate when drying onto the conjugate pad. 10 ul of sample containing different amount of celecoxib is applied to the test trip and chased with 80-90 ul of chasing buffer. Assay time is 15-20 mins before taking the reading.

Reader Output: Intensity vs Position. Readout of the results of scanning the test strips was generated using Qiagen reader (Qiagen, Germany). The intensity (peak area) of the test line(s) and control line is measured and the ratio of Testline/Control (T/C) line was calculated.

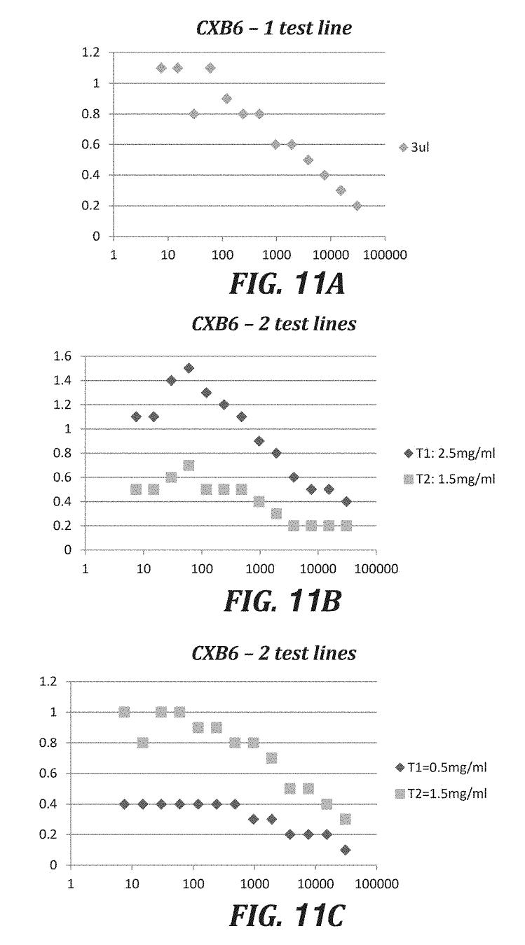

Standard Curve. Standard curves of ratio of Testline/Control (T/C) vs. celecoxib concentration were generated in the “liquid phase” format and are illustrated in FIGS. 11A-13C.

FIG. 11A illustrates the standard curve, i.e., the ratio of test line over control line (T/C) vs. celecoxib concentration for the lateral flow assay (LFA) using the anti-celecoxib monoclonal antibody CXB6. In this assay, a single test (T) line of BSA-celecoxib was striped at 0.7 mg/ml. 3 μl of 8 μg/ml CXB6 mAb/colloidal gold conjugate (pH 7.5; OD5) was applied onto the conjugate pad. FIG. 11B and 11C illustrate the standard curves for an LFA using two test lines (T1 and T2) of the anti-celecoxib monoclonal antibody CXB6. T1 was striped at 2.5 mg/ml BSA-celecoxib (FIG. 4B) and 0.5 mg/ml BSA-celecoxib (FIG. 11C). T2 was striped at 1.5 mg/ml BSA-celecoxib (FIG. 11B and FIG. 11C). 5 μl of 8 μg/ml CXB6 mAb/colloidal gold conjugate (pH 7.5; OD10) was applied onto the conjugate pad. Both assays illustrate that the anti-celecoxib monoclonal antibody CXB6 exhibited high sensitivity for celecoxib with detectable binding at the T (or T1) line reduced only at higher concentrations of competing celecoxib spiked into the flow. The large difference in T/C ratio between the T1 and T2 lines observed in the two line test (FIG. 11B and FIG. 11C) demonstrates a much higher sensitivity for the antibody when placed closer to the sample port, where concentration of analyte is likely to be higher.

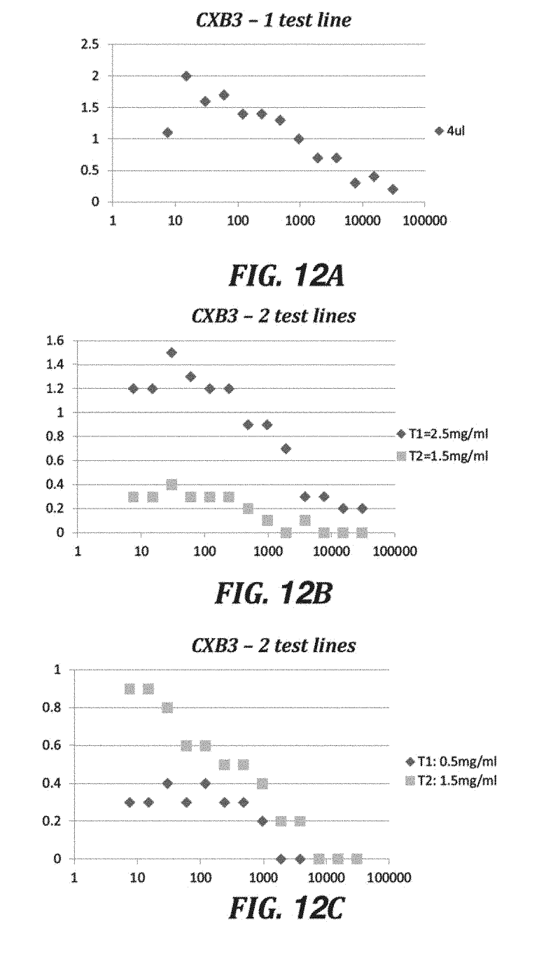

FIG. 12A illustrates the standard curve, i.e., the ratio of test line over control line (T/C) vs. celecoxib concentration for the lateral flow assay (LFA) using the anti-celecoxib monoclonal antibody CXB3. In this assay, a single test (T) line of BSA-celecoxib was striped at 0.7 mg/ml. 4 μl of 4 μg/ml CXB3 mAb/colloidal gold conjugate (pH 7.5; OD9) was applied onto the conjugate pad. FIGS. 12B and 12C illustrate the standard curves for an LFA using two test lines (T1 and T2) of the anti-celecoxib monoclonal antibody CXB3. T1 was striped at 2.5 mg/ml BSA-celecoxib (FIG. 12B) and 0.5 mg/ml BSA-celecoxib (FIG. 12C). T2 was striped at 1.5 mg/ml BSA-celecoxib (FIG. 12B and FIG. 12C). 5 μl of 6 μg/ml CXB3 mAb/colloidal gold conjugate (pH 7.5; OD6 or 8) was applied onto the conjugate pad. Both assays illustrate that the anti-celecoxib monoclonal antibody CXB3 exhibited high sensitivity for celecoxib with detectable binding at the T (or T1) line reduced only at higher concentrations of competing celecoxib spiked into the flow. The large difference in T/C ratio between the T1 and T2 lines observed in the two line test (FIG. 12B and FIG. 12C) demonstrates a much higher sensitivity for the antibody when placed closer to the sample port, where concentration of analyte is likely to be higher.

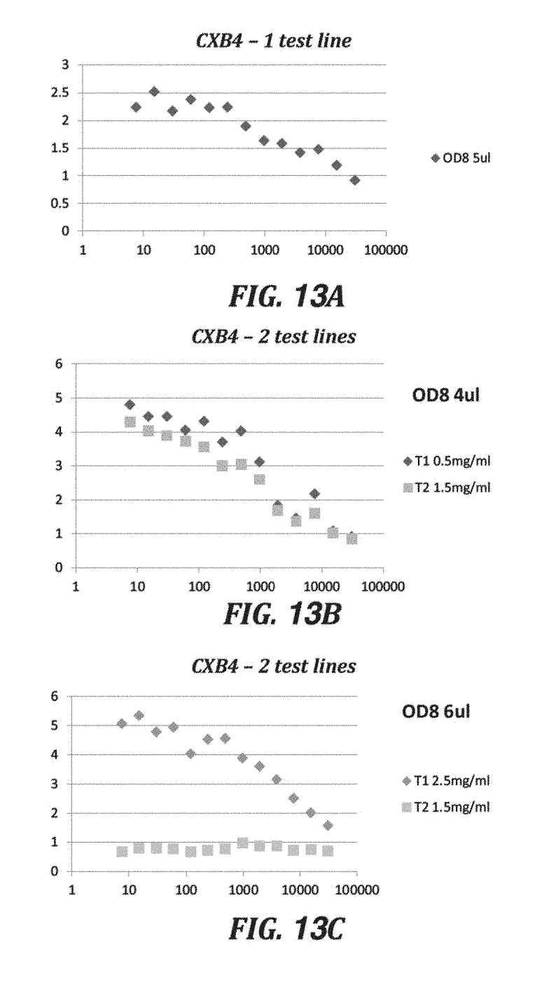

FIG. 13A illustrates the standard curve, i.e., the ratio of test line over control line (T/C) vs. celecoxib concentration for the lateral flow assay (LFA) using the anti-celecoxib monoclonal antibody CXB4. In this assay, a single test (T) line of BSA-celecoxib was striped at 0.5 mg/ml. 5 μl of 4 μg/ml CXB3 mAb/colloidal gold conjugate (pH 7.5; OD8) was applied onto the conjugate pad. FIGS. 13B and 13C illustrate the standard curves for an LFA using two test lines (T1 and T2) of the anti-celecoxib monoclonal antibody CXB4. T1 was striped at 2.5 mg/ml BSA-celecoxib (FIG. 13C) and 0.5 mg/ml BSA-celecoxib (FIG. 13B). T2 was striped at 1.5 mg/ml BSA-celecoxib (FIG. 13B and FIG. 13C). 6 μl of 4 μg/ml CXB4 mAb/colloidal gold conjugate (pH 7.5; OD8) (FIG. 13C) and 4 μl of 4 μg/ml CXB4 mAb/colloidal gold conjugate (pH 7.5; OD8) (FIG. 13B) was applied onto the conjugate pad. Both assays illustrate that the anti-celecoxib monoclonal antibody CXB4 exhibited high sensitivity for celecoxib with detectable binding at the T (or T1) line reduced only at higher concentrations of competing celecoxib spiked into the flow. The large difference in T/C ratio between the T1 and T2 lines observed in the two line test (FIG. 13B and FIG. 13C) demonstrates a much higher sensitivity for the antibody when placed closer to the sample port, where concentration of analyte is likely to be higher.

While illustrative embodiments have been illustrated and described, it will be appreciated that various changes can be made therein without departing from the spirit and scope of the disclosure.

While the preferred embodiment of the invention has been illustrated and described, it will be appreciated that various changes can be made therein without departing from the spirit and scope of the invention.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description.

The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A lateral flow assay device, comprising:
(a) a sample receiving zone for receiving the liquid sample;
(b) a detection reagent zone in liquid communication with the sample receiving zone and downstream in flow direction from the sample receiving zone; wherein the detection reagent zone comprises a detection reagent deposited thereon, and wherein the detection reagent is an celecoxib antibody, or fragment or derivative thereof that binds celecoxib, labeled with a detectable reporting group; and
(c) a capture zone in liquid communication with the detection reagent zone and downstream in flow direction from the detection reagent zone; wherein the capture zone comprises first and second capture reagents immobilized thereon, the first capture reagent positioned upstream in flow direction from the second capture reagent, wherein the first capture reagent is a celecoxib antigen capable of binding the detection reagent, and wherein the second capture reagent is an antibody capable of binding the detection reagent.

2. The device of claim 1, wherein the detectable reporting group is selected from colloidal gold, latex particles, colored dyes, paramagnetic particles, and fluorescent particles.

3. The device of claim 1, wherein the celecoxib antigen is a celecoxib-protein conjugate.

4. The device of claim 1, wherein distance between the sample receiving zone and the first capture reagent is varied or minimized to optimize celecoxib detection sensitivity.

5. The device of claim 1, further comprising a third capture zone intermediate the first and second capture zones, wherein the third capture zone comprises a celecoxib antigen capable of binding the detection reagent.

6. The device of claim 1, wherein two or three lines can be used to generate multiple readings on the same sample allowing for increase reproducibility and expanded dynamic range.

7. A method for assaying celecoxib, comprising
(a) applying a liquid sample comprising celecoxib (e.g., subject's blood sample) to a lateral flow assay device, the device having
(i) a sample receiving zone for receiving the liquid sample;
(ii) a detection reagent zone in liquid communication with the sample receiving zone and downstream in flow direction from the sample receiving zone; wherein the detection reagent zone comprises a detection reagent deposited thereon, and wherein the detection reagent is an celecoxib antibody, or fragment or derivative thereof that binds celecoxib, labeled with a detectable reporting group; and
(iii) a capture zone in liquid communication with the detection reagent zone and downstream in flow direction from the detection reagent zone; wherein the capture zone comprises first and second capture reagents immobilized thereon, the first capture reagent positioned upstream in flow direction from the second capture reagent, wherein the first capture reagent is a celecoxib antigen capable of binding the detection reagent, and wherein the second capture reagent is an antibody capable of binding the detection reagent;
(b) allowing the sample to flow from the sample receiving zone through the detection reagent zone to provide a detection reagent with celecoxib (e.g., combination of detection agent with bound celecoxib, optionally free detection reagent, and optionally free celecoxib);
(c) allowing the detection reagent with celecoxib to flow through the capture zone, whereby the first capture reagent binds free detection reagent to provide detection reagent bound to the first capture reagent, and whereby the second capture reagent binds detection reagent with or without bound celecoxib; and
(d) observing the amount of detection reagent bound to the first capture reagent relative to the second capture reagent.

8. The method of claim 7 further comprising determining the quantity of celecoxib in the sample by quantitating the amount of detection reagent bound at control line and test line.

9. The method of claim 7, wherein quantitating the amount of detection reagent bound to the capture reagents comprises optical density measurement.

10. The method of claim 7, wherein the detectable reporting group is selected from colloidal gold, latex particles, colored dyes, paramagnetic particles, and fluorescent particles.

11. The method of claim 7, wherein the celecoxib antigen is a celecoxib protein conjugate.

12. The method of claim 7, wherein distance between the sample receiving zone and the first capture reagent is varied to optimize celecoxib detection sensitivity.

13. The method of claim 7, wherein distance between the sample receiving zone and the first capture reagent is minimized to optimize celecoxib detection sensitivity.

14. The method of claim 7, further comprising observing the amount of excess detection reagent bound to the second capture reagent (control line).

15. The method of claim 7, further comprising determining the quantity of celecoxib in the sample by quantitating the amount of excess detection reagent bound to the second capture reagent.

16. The method of claim 7, further comprising a third capture zone intermediate between the first and second capture zones, wherein the third capture zone comprises a celecoxib antigen capable of binding the detection reagent.

17. The method of claim 16, comprising determining the quantity of celecoxib in the sample by quantitating the amount of detection reagent bound to the third capture reagent.

18. The method of claim 17, wherein quantitating the amount of detection reagent bound to the third capture reagent comprises optical density measurement.

19. The method of claim 7, wherein two or three lines can be used to generate multiple readings on the same sample allowing for increase reproducibility and expanded dynamic range.

20. A method for improving the effectiveness of osteopathic pain therapy by monitoring a subject's compliance by determining one or more pharmacokinetic parameters of the subject with a point-of-care device after administration of a pain drug, the method comprising:
(a) administering a pain drug (e.g., celecoxib) at a first dose to a subject in need of osteopathic pain therapy;
(b) determining the concentration of the pain drug the subject's blood at one or more time points after administration to provide a set of pain drug concentration/time data points, wherein the determination of the concentration of the pain drug is made using the device of claims 1;
(c) transforming the set of pain drug concentration/time data points to provide one or more pharmacokinetic parameters; and
(d) administering the pain drug at subsequent doses (e.g., second and subsequent doses) to achieve a target optimal value for the one or more pharmacokinetic parameters.

21. The method of claim 20, wherein the one or more pharmacokinetic parameters are selected from the group consisting of time to maximum concentration (Tmax), concentration maximum (Cmax), area under the curve (AUC), clearance (CL), volume of distribution (Vd), apparent volume of distribution during the terminal phase (Vz), apparent volume of distribution during steady state (Vss) and combinations thereof.

22. The method of claim 21, wherein the one or more pharmacokinetic parameters is area-under-the-curve (AUC).

23. The method of claim 20, wherein the pain drug is a COX-2 inhibitor.

24. The method of claim 23, wherein the pain drug is COX-2 fixed dose combination (FDC) where the other component can either be diuretic, ARB such as olmesartan, or ACE inhibitor such as lisinopril, or hydrochlorothiazide (HCTZ).

25. The method of claim 23, wherein the pain drug is celecoxib.

26. The method of claim 20, wherein the subject is in need of treatment for osteopathic pain, and the method comprises administration of celecoxib FDC.

27. The method of claim 20, wherein the subject is in need of treatment for osteopathic pain, and the method comprises administration of a single dosage form that comprises celecoxib and another drug.

28. The method of claim 27, wherein single dosage form comprises celecoxib and HCTZ or lisinopril or olmesartan.

29. The method of claim 20, wherein the pain is osteopathic pain.

30. A monoclonal antibody produced by a hybridoma CXB3, CXB4 or CXB6, accession numbers ______.
